(19) United States
(12) Patent Application Publication
(10) Pub. No.: US 2025/0143880 A1
(43) Pub. Date: May 8, 2025

(54) DEVICE, SYSTEM, AND METHOD FOR REDUCING MITRAL VALVE REGURGITATION

(71) Applicant: TAU MEDICAL INC., Busan (KR)

(72) Inventor: June Hong Kim, Busan (KR)

(21) Appl. No.: 19/017,253

(22) Filed: Jan. 10, 2025

Related U.S. Application Data
(62) Division of application No. 17/764,870, filed on Mar. 29, 2022.
(60) Provisional application No. 63/086,710, filed on Oct. 2, 2020.
(60) Provisional application No. 62/909,929, filed on Oct. 3, 2019.

(51) Int. Cl. A61F 2/24; A61B 17/12

(52) U.S. Cl. CPC A61F 2/2451; A61F 2/2466; A61B 17/12009; A61F 2220/0091

(57) ABSTRACT

A device for reducing mitral valve regurgitation comprises a first protective tube and a second protective tube. Proximal portions of the two protective tubes are being attached side-by-side along at least a portion of the length of the two protective tubes to define a stem portion. Distal portions of the two protective tubes are being separated thereafter to define a hinge portion. The MLC device has a stopper being attached on the distal end of the second protective tube to configure to prevent further advancement of the second protective tube into heart muscle. The first protective tube has at least one anchor disposed between the hinge portion and the distal end of the first protective tube. The anchor configured to lodge into a coronary sinus and maintain the tissue protective device in place.

The present application is a divisional of U.S. application Ser. No. 17/764,870, filed Mar. 29, 2022; which is a 371 National Stage Entry of International Application No. PCT/IB2020/059290, filed Oct. 2, 2020 (now published as WO2021064689); which claims the benefit of U.S. Provisional Application No. 63/086,710, filed Oct. 2, 2020, and U.S. Provisional Application No. 63/909,929, filed Oct. 3, 2019; all of which are incorporated herein in their entirety and referenced thereto.

### TECHNICAL FIELD

The disclosure relates to device, system, and method for reducing cardiac valve regurgitation and, more particularly, to transcatheter device, system, and method for treatment for mitral valve regurgitation.

### BACKGROUND

Mitral regurgitation is characterized by cardiomyopathy, mitral annular enlargement, and leaflet traction contributing to malcoaptation. A transcatheter mitral loop cerclage (“MLC”) device applies a circumferential compression to the mitral annulus by creating a loop through the coronary sinus across the interventricular septum. There is a need to have the mitral loop cerclage device to be able to maintain its position from being pushed back into the coronary sinus.

### SUMMARY

The MLC device for reducing mitral valve regurgitation comprises a first protective tube and a second protective tube. The MLC device has a cerclage rope disposed within the first protective tube and the second protective tube. The first protective tube and the second protective tube each has a proximal end and a distal end. Proximal portions of the two protective tubes are being attached side-by-side along at least a portion of the length of the two protective tubes to define a stem portion. Distal portions of the two protective tubes are being separated thereafter to define a hinge portion.

The MLC device has a stopper being attached on the distal end of the second protective tube to configure to prevent further advancement of the second protective tube into heart muscle. The cerclage rope has an arch portion arranged between the distal ends of the two protective tubes. The first protective tube has at least one anchor disposed between the hinge portion and the distal end of the first protective tube. The anchor configured to lodge into a coronary sinus and maintain the tissue protective device in place.

The first protective tube is configured to be inserted through a coronary sinus to encircle at least part of a mitral valve. The first protective tube has a preformed shape in such as way the first tube is configured to maintain the preformed shape when the first tube traverses through a coronary sinus.

The second protective tube is configured to pass through a tricuspid valve and rest against the right ventricular side of the interventricular septum. The second protective tube has a preformed shape in such as way the second tube is configured to maintain the preformed shape when the second tube traverses through the tricuspid valve. The second protective tube is rigid enough to resist being bent inward as tension is applied.

The method of treating mitral valve regurgitation in a patient by using the MLC device comprises (a) inserting a sheath introducer in a femoral vein; (b) inserting a guide catheter to engage in a coronary sinus through a femoral sheath; (c) traversing a guidewire into an interventricular septum and exiting into the right ventricular (RV) cavity; (d) placing a guidewire catcher in the right ventricular cavity through the femoral sheath and grabbing the exiting guidewire; (e) pulling the guidewire out to the femoral sheath with the guidewire catcher so that the guidewire makes up a path of the femoral vein: inferior vena cava (IVC)-coronary sinus (CS)-right ventricular (RV) cavity-right atrium (RA)-inferior vena cava (IVC)-femoral vein; (f) connecting a proximal end of the guidewire to a cerclage rope with a tension lock device; (g) replacing the guidewire with the cerclage rope in place by pulling a distal end of the guidewire; (h) advancing an MLC device through the femoral sheath over the cerclage rope with a delivery system; (i) when the MLC device is on optimal tension, securing an anchor to keep the MLC device in place; (j) when the MLC device is in place, delivering a suitable tension through the tension lock device under a guidance of real time imaging such as echocardiogram; (k) cutting the cerclage rope by a cutting device.

### BRIEF DESCRIPTION OF THE DRAWINGS

Features and advantages of the claimed subject matter will be apparent from the following description of embodiments consistent therewith, which description should be considered in conjunction with the accompanying drawings, wherein:

FIG. 1 depicts an embodiment of the MLC device which includes a stem portion, a first tube, and a second tube;

FIG. 2 depicts an embodiment of the MLC device which shows a hinge portion and a stopper on the second arm;

FIG. 3 depicts an embodiment of the MLC device which shows a lumen of the stopper and a lumen of the first arm;

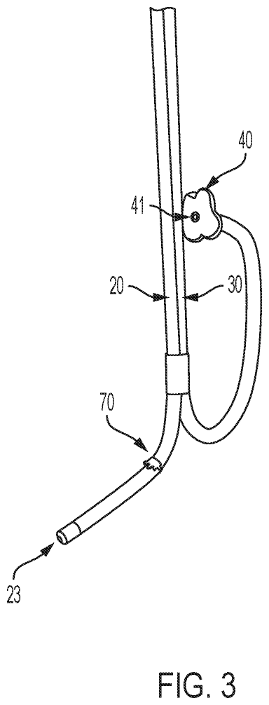

FIG. 4 depicts an embodiment of a cerclage rope which includes an arch portion and an outer layer;

FIG. 5 depicts an embodiment of a cerclage rope which includes an arch portion and an outer layer;

FIG. 6 depicts an embodiment of the MLC device when the MLC device was advanced over the cerclage rope;

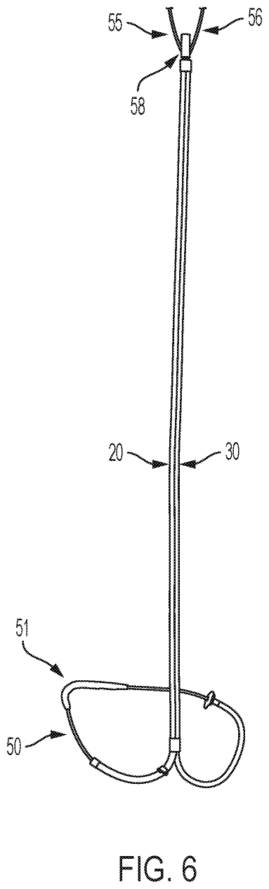

FIG. 7 depicts an embodiment of the MLC device when the MLC device was advanced over the cerclage rope;

FIG. 8 depicts an embodiment of the MLC device when the MLC device was advanced over the cerclage rope;

FIG. 9 depicts an embodiment of the MLC device how the MLC device is advanced over the cerclage rope;

FIG. 10A depicts a procedure where coronary venography through a balloon wedge catheter identifies a basal septal perforator coronary vein through which a guidewire is advanced;

FIG. 10B depicts a procedure where a target-capture device is positioned into the right ventricular outflow tract to ensnare the guidewire that crossed from the coronary vein.

FIG. 10C depicts a procedure where the guidewire is exchanged for tension device containing an integrated coronary artery protection element.

FIG. 10D depicts a procedure where during application of tension, the protection element prevents compression of entrapped circumflex coronary artery branches;

FIG. 10E depicts a procedure where in this caudal left anterior oblique projection, the mitral loop cerclage system is shown to surround the mitral annular plane;

FIG. 10F depicts a procedure where the tension locking device was embedded in the left subclavicular pocket;

FIG. 11 shows the tension locking device is embedded in the left subclavicular pocket;

FIG. 12 depicts an embodiment of MLC device when the tension is loose;

FIG. 13 depicts an embodiment of MLC device when the tension is applied;

FIG. 14 depicts the stopper is placed on the wall of the IVS;

FIG. 15 depicts the first arm encircles the mitral valve;

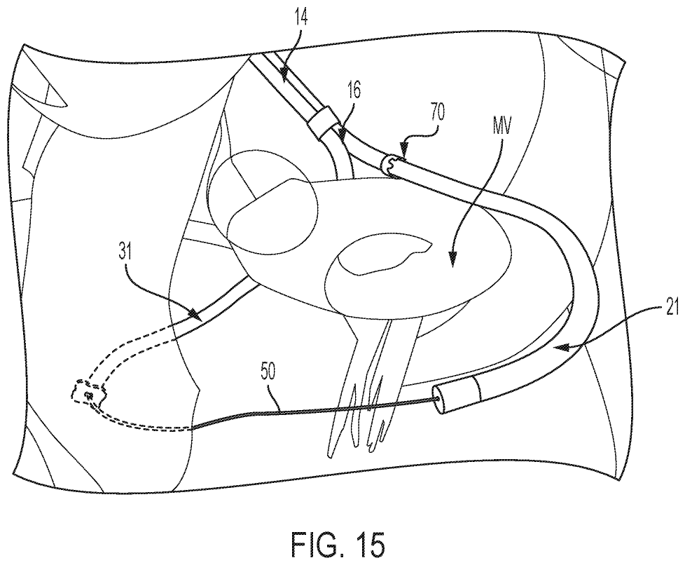

FIG. 16 depicts the anchor is placed on the first arm;

FIG. 17 depicts the anchor is placed on the first arm;

FIG. 18 depicts the anchor is placed on the first arm;

FIG. 19 depicts an embodiment of the anchor;

FIG. 20 depicts the anchor is placed in the coronary sinus;

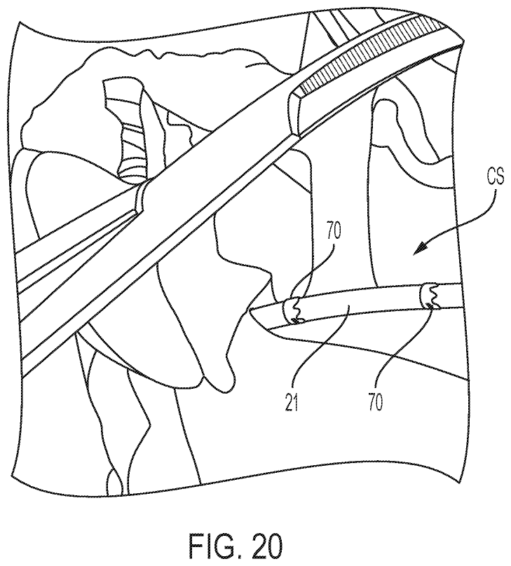

FIG. 21 depicts preferred anchoring sites; and

FIG. 22 depicts preferred and other possible anchoring sites. DISCLOSURE

In the drawings, like numerals indicate like elements throughout. Certain terminology is used herein for convenience only and is not to be taken as a limitation on the present invention. The terms “distal” and “proximal” refer to the directions “away from” and “closer to,” respectively, the body of the physician inserting the introducer sheath into a patient. The terminology includes the words above specifically mentioned, derivatives thereof, and words of similar import.

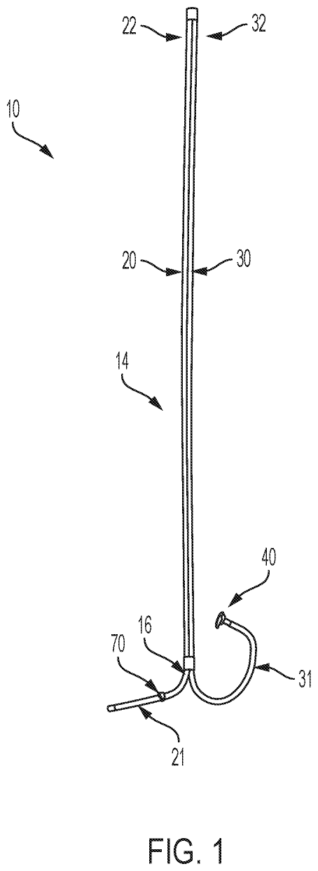

The mitral loop cerclage (“MLC”) device 10 may comprise a first tube 20 and a second tube 30 as shown in FIG. 1. The first tube 20 may be configured to engage with coronary sinus (CS). The second tube 30 may be configured to engage with tricuspid valve (TV).

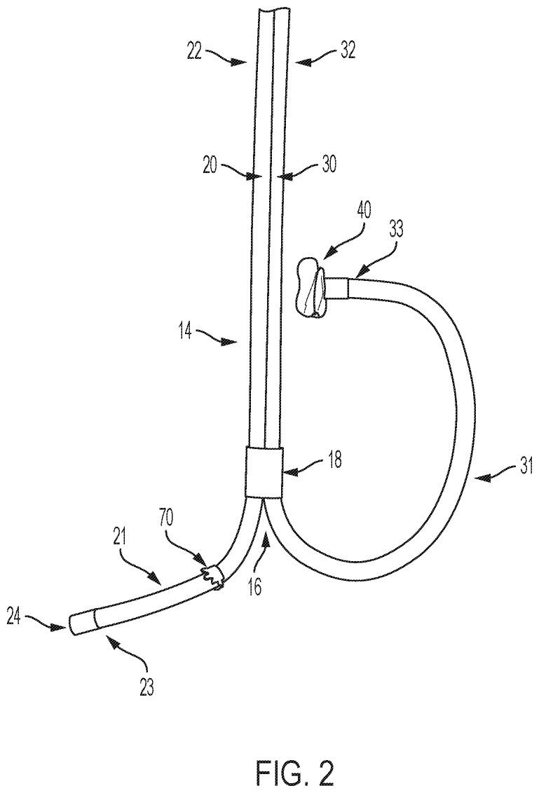

As shown in FIG. 2, the first tube 20 has a proximal end 22 and a distal end 23, and a lumen 24 expanding. The second tube 30 also has a proximal end 32 and a distal end 33, and a lumen 34 expanding therebetween. The proximal portions of the first tube 20 and the second tube 30 may be attached side-by-side longitudinally along at least a portion of the length of the two tubes 20, 30 to define a stem portion 14, and the distal portions of the first and second tubes 20, 30 may be separated thereafter to define a hinge portion 16. The first tube 20 and the second tube 30 may be formed of soft materials like synthetic, rubber, soft plastic, etc., or metal materials such as a coil spring, etc.

### Stem Portion

As shown in FIGS. 1-2, the MLC device 10 may include the stem portion 14. The proximal portions of the first tube 20 and the second tube 30 may be attached side-by-side longitudinally and then separate from each other distally. The attached portion between the first and second tubes 20, 30 is defined as a stem portion 14. The portion where the two tubes 20, 30 start to separate is defined as a hinge portion 16. The length of the stem portion 14 may vary depending on the need.

The role of the stem portion 14 is to stabilize the first tube 20 and the second tube 30 so that their positions may be maintained. When the hinge portion 16 rests on the orifice of the coronary sinus, the MCL device 10 is prevented from advancing into the coronary sinus nor the tricuspid valve. HINGE PORTION

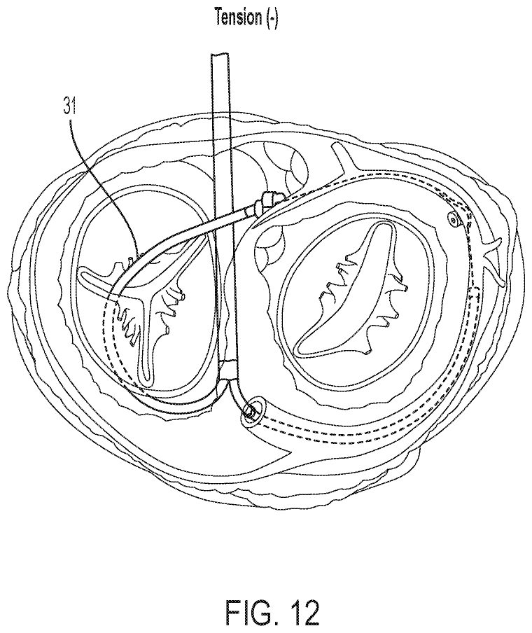

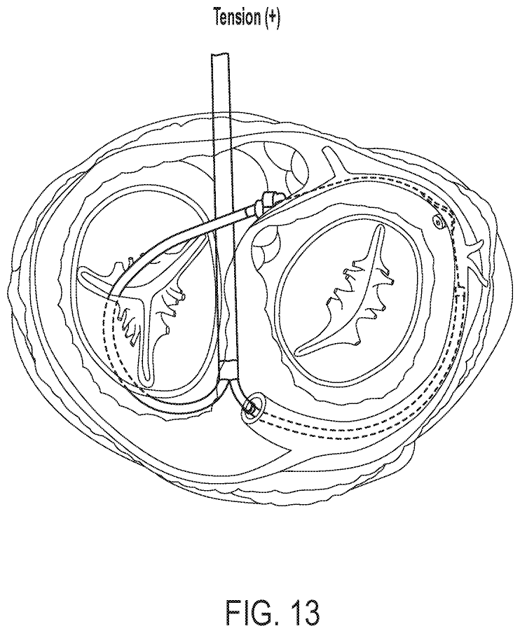

The MLC device 10 may include the hinge portion 16 as shown in FIGS. 1-2. The hinge portion 16 is defined as the portion where the first tube 20 and the second tube 30 start to separate from each other. The hinge portion 16 may be placed at or near the orifice of the coronary sinus as shown in FIGS. 12-13.

The role of the hinge portion 16 is to stabilize the first tube 20 and the second tube 30 so that their positions may be maintained. When the hinge portion 16 rests on the orifice of the coronary sinus, the MCL device 10 is prevented from advancing into the coronary sinus nor the tricuspid valve.

### First Arm for Mitral Valve

The first tube 20 may include the lumen 24 extending between the proximal end 22 and distal end 23. As shown in FIG. 2, the first arm portion 21 of the first tube 20 is defined as a portion from the hinge portion 16 to the distal end 23. As shown in FIGS. 1-2, the first arm portion 21 may be pre-shaped with soft materials like synthetic, rubber, soft plastic, etc., or metal materials such as a coil spring, etc. such that it maintains the preformed shape when traverses through the coronary sinus. FIGS. 12-13 show the first arm portion 21 traversing through the coronary sinus.

The first arm portion 21 is configured to be inserted through the coronary sinus to encircle at least part of the mitral valve. When a tension is applied, as shown FIG. 13, the first arm portion 21 is configured to tighten the annulus of the mitral valve for reducing the regurgitation.

When the cerclage rope 50 is inserted into the MLC device 10, the first arm portion 21 prevents direct contact of the cerclage rope 50 and protects the coronary sinus and its surrounding tissues from damage. The length of the first arm portion 21 may vary as needed from patient to patient. The length may be determined based on estimation from prior imaging of individual patient.

### Second Arm for Tricuspid Valve

The second tube 30 may include a lumen 34 expanding between the proximal end 32 and distal end 33. As shown in FIG. 2, the second tube 30 includes a second arm portion 31 which is defined as the portion between the hinge portion 16 and the distal end 33 of the second tube 30.

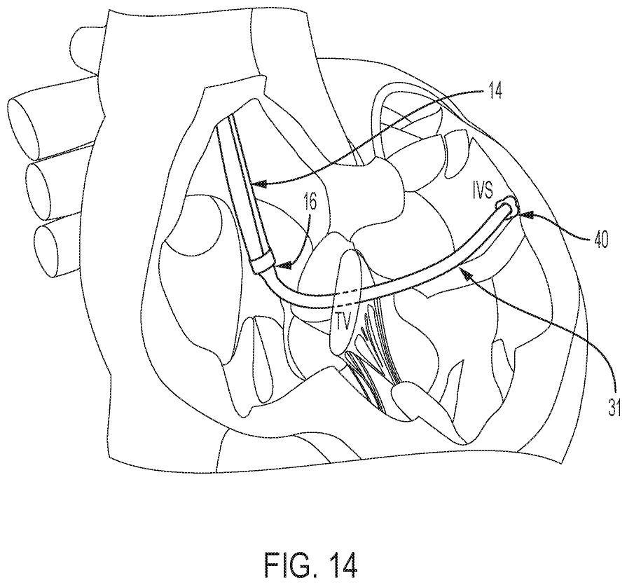

The second arm portion 31 is configured to pass through the tricuspid valve. As shown in FIGS. 1-2, the second arm portion 31 may be pre-shaped with soft materials like synthetic, rubber, soft plastic, etc., or metal materials such as a coil spring, etc. such that it maintains the preformed shape when it passes through the tricuspid valve. The second arm portion 31 may be rigid enough to resist being bent as tension is applied onto the cerclage rope 54. FIGS. 12-14 show the second arm portion 31 passing through the tricuspid valve. The length of the second arm portion 31 may vary as needed from patient to patient. The length may be determined based on estimation from prior imaging of individual patient.

### Stopper

The second tube 30 also includes a stopper 40. The stopper 40 may be attached to the distal portion of the second tube 30. Preferably, the stopper 40 may be securely attached at the distal end 33 of the second tube 30 as shown in FIGS. 1-2. The stopper 40 is configured to rest against the right ventricular side of the interventricular septum as shown in FIGS. 12-14. The stopper 40 is configured to keep the second arm portion 31 maintain its preformed shape and position by resting against the interventricular septum. The stopper 40 also keeps the second arm portion 31 afloat rather than in direct contact with the tricuspid valve as tension is applied on the cerclage rope 50. Further, the stopper 40 prevents the distal end 33 of the second tube 30 from advancing into the interventricular septum.

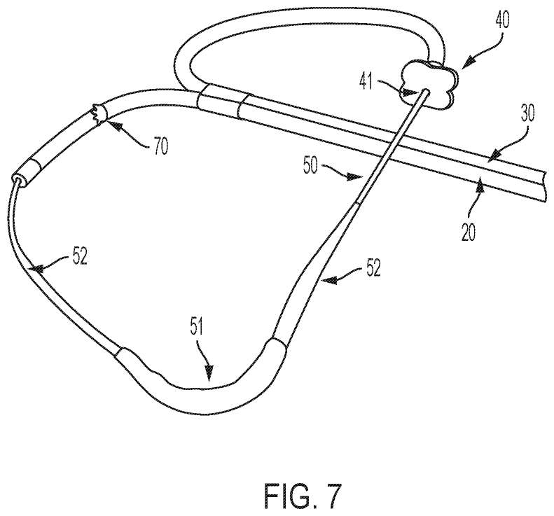

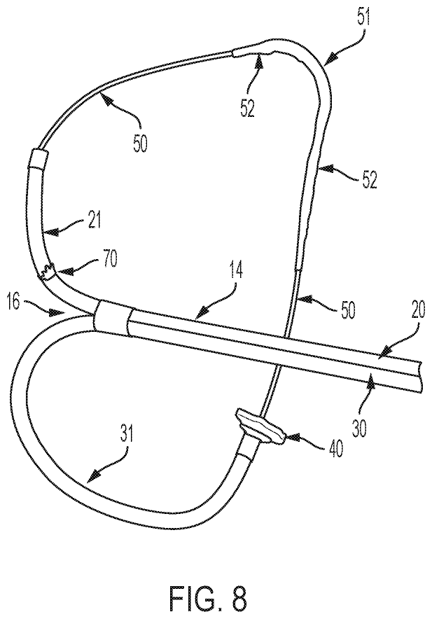

As shown in FIGS. 7-8, the stopper 40 may include a lumen 41 in the middle where the cerclage rope 50 may traverse through the lumen. The size, shape and volume of the stopper 41 may vary as needed from patient to patient, however, it should be larger in diameter than the second arm portion 31. Those skilled in the art will recognize that the stopper 40 may have a shape other than the disk as shown in FIG. 7.

### Cerclage Rope for Tension

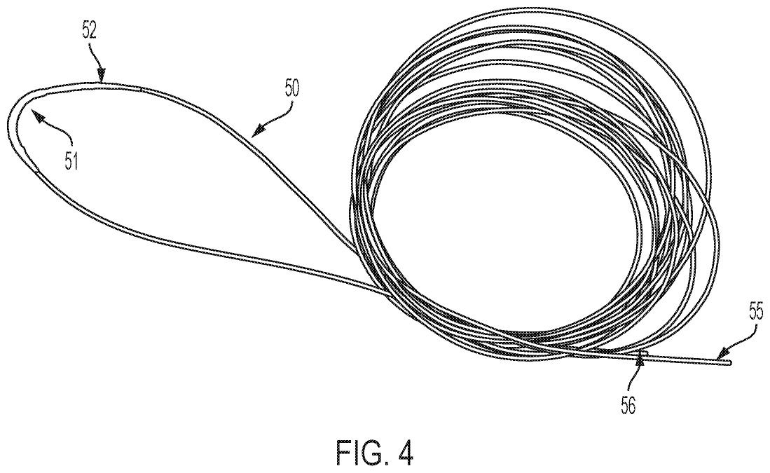

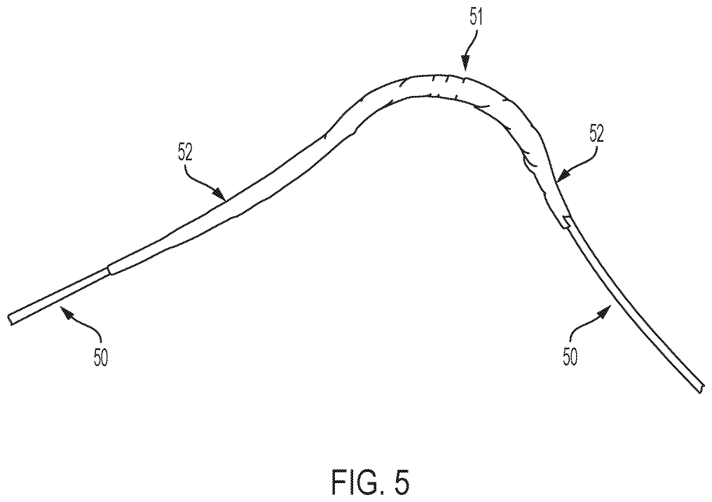

FIG. 4 shows the cerclage rope 50. The cerclage rope 50 comprises an arch portion 51. The arch portion 51 is covered by an outer layer 52 as shown on FIGS. 4-5. The outer layer 52 is coated integrally onto the arch portion 51. Preferably, the outer layer may be coated longer and tapered towards the stopper 40 as shown on FIG. 8. The arch portion 51 and its outer layer 52 is configured to protect the coronary artery and its surrounding heart tissues as tension is applied onto the cerclage rope 50.

The cerclage rope 50 may be made of synthetic materials such as nylon, metal (i.e., stainless steel) or metals coated with nylon, etc. The coating portion 52 may be made of biocompatible synthetic resin. ANCHOR

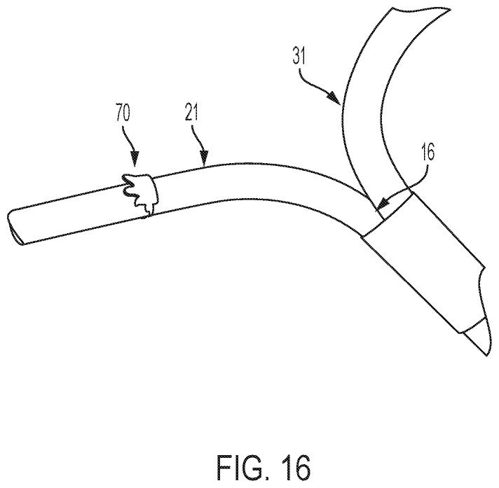

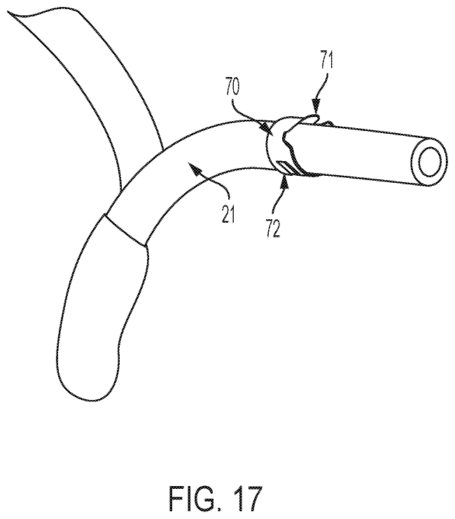

The preferred embodiment of the MLC device 10 further comprises an anchor 70. As shown in FIGS. 16-17, the anchor 70 is preferably positioned on the first arm portion 21 close to the hinge portion 16. The anchor 70 can also be positioned at other locations along the first arm portion 21.

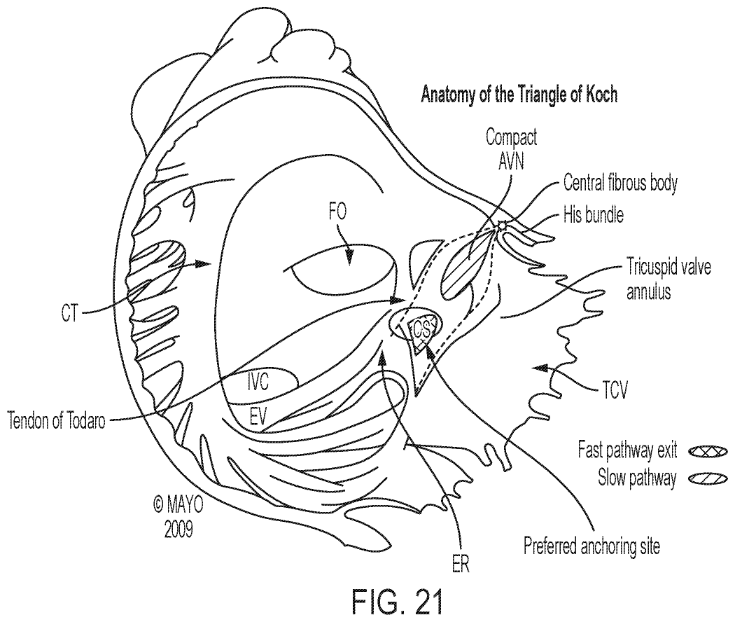

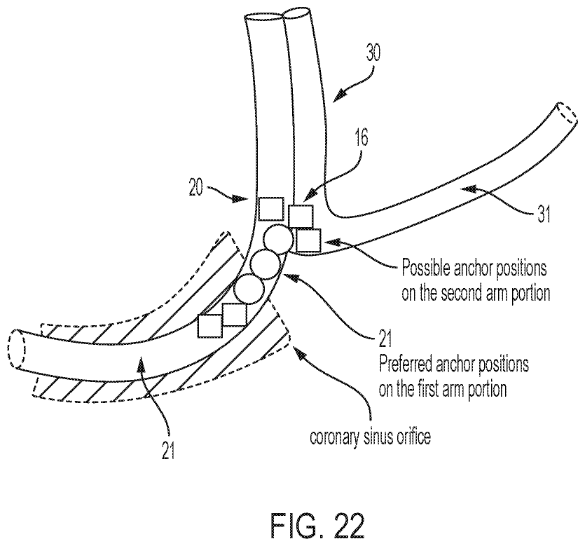

FIGS. 21-22 shows a plurality of the anchors 40 positions along either the first arm portion 21 and/or the second arm portion 31. The preferred anchor 40 positions are marked as circles along the first arm portion 21 closes to the hinge portion 16. Other possible anchor locations on the first and second arm portions 21, 31 are marked as squares. When tension may be applied by the cerclage rope 50, the anchor 70 is configured to maintain the MLC device 10 in place and prevent further advancement of the device 10 into the coronary sinus.

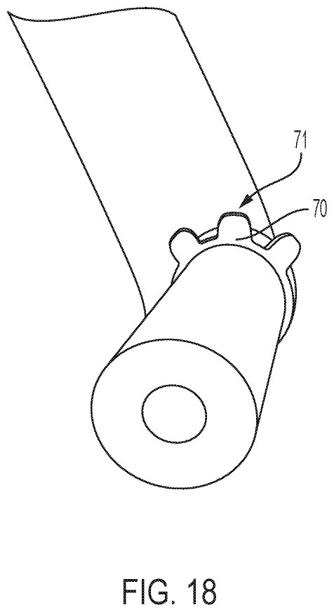

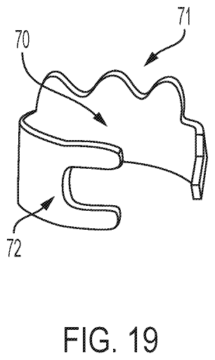

As shown in FIGS. 17-19, the anchor 70 may include at least one anchor head 71 and at least one anchor arm 72. The anchor head 71 is configured to lodge into the preferred anchoring sites as shown in FIG. 21. The anchoring arm 72 is configured to be securely attached on the first arm portion 21 and/or the second arm portion 31. A plurality of the anchor 70 may be securely attached on the first arm portion and/or second arm portion 31.

### Mitral Loop Cerclage (MLC) Procedure

Under moderate sedation (n=3) or general anesthesia (n=2), 19-F<c>15-cm introducer sheaths (Oscor, Palm Harbor, Florida) may be placed both in the left subclavian vein (via a pacemaker-like pocket) and a femoral vein. This may allow the operator to stand at the right groin. Transesophageal echocardiography (TEE) or transthoracic echocardiography may be used under anesthesia or sedation, respectively, to adjust tension interactively to reduce MR. Heparin may be administered to achieve an activated clotting time of >300 s.

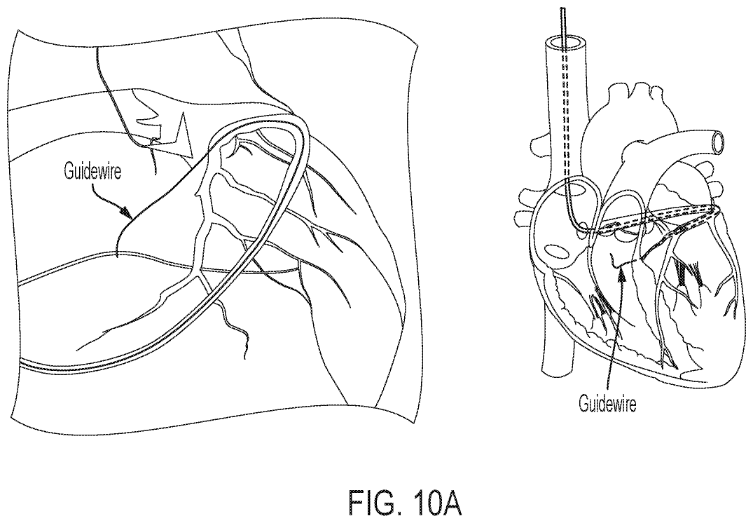

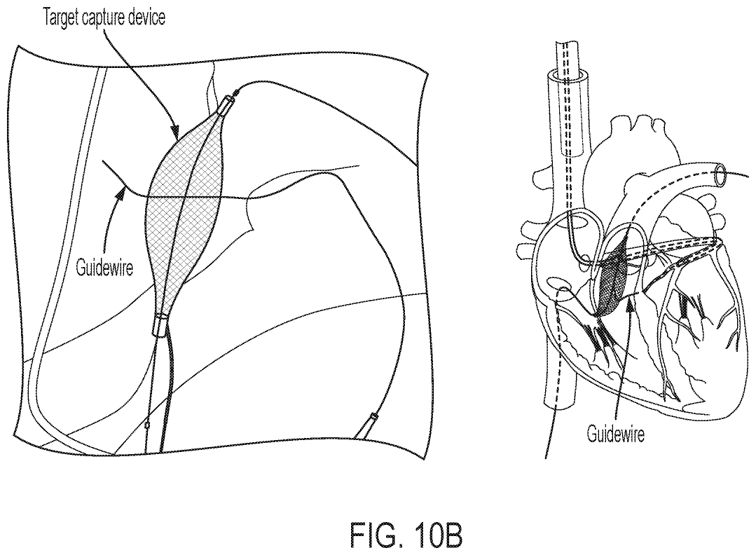

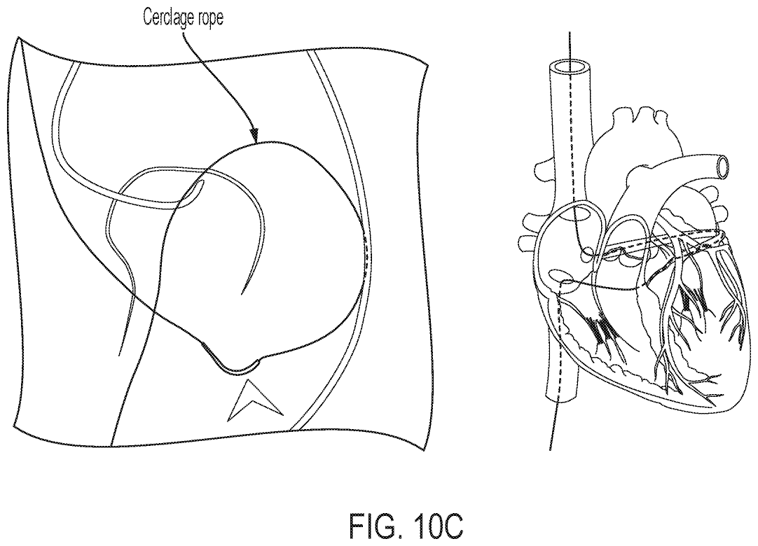

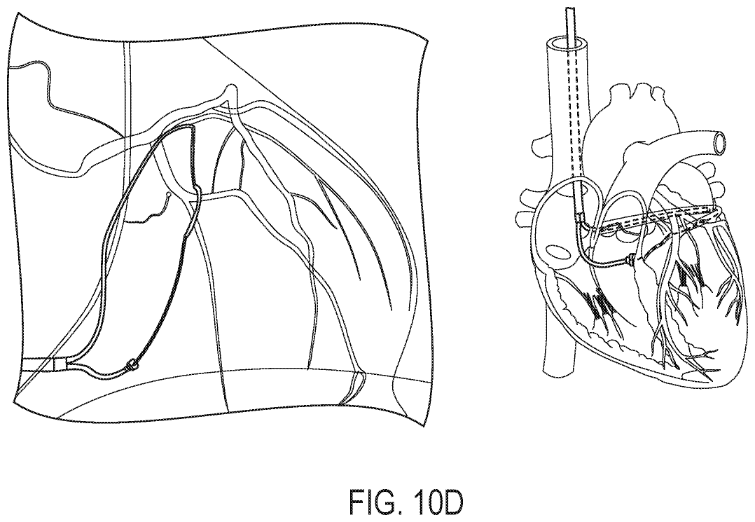

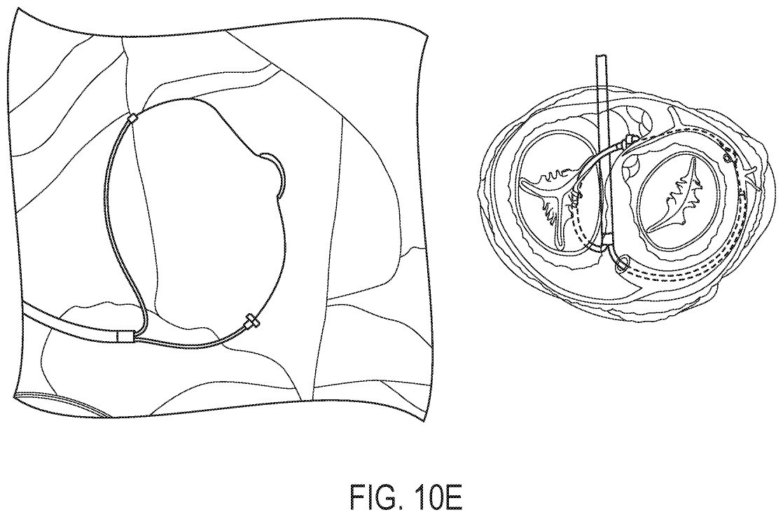

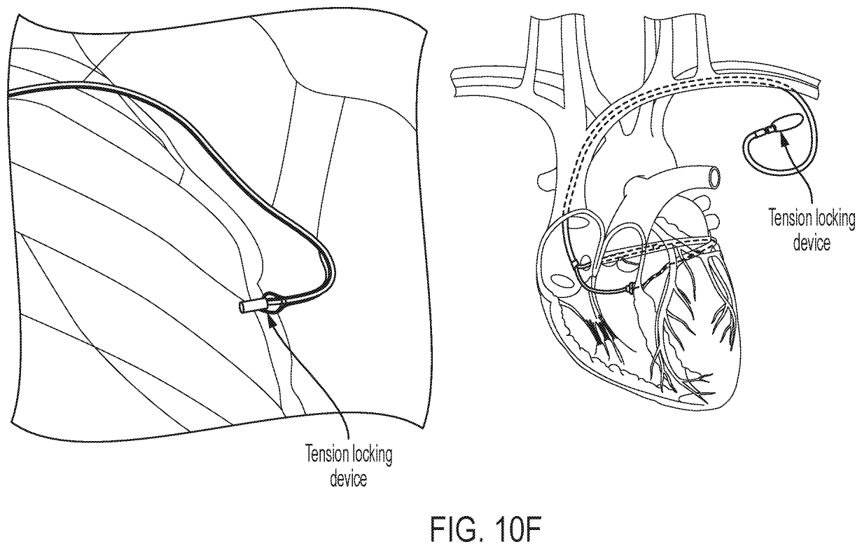

A balloon-tip coronary sinus guiding catheter (Cello, Medtronic) may be placed from the left subclavian vein into the great cardiac vein and a pressurized contrast venogram performed. Through the coronary sinus guide, a dual-lumen 0.014-inch microcatheter (Crusade, Kaneka Medix, Osaka, Japan) may be positioned in the basal septal perforator coronary vein over a soft 0.014-inch guidewire (Whisper, Abbott, Chicago, Illinois) and then used to direct a stiff-tip 0.014-inch peripheral guidewire (Astato XS 20, Asahi, Japan) to traverse the interventricular septum into the RVOT. As shown in FIG. 10A, coronary venography through a balloon wedge catheter may identify a basal septal perforator coronary vein (arrowhead) through which a guidewire may be advanced.

A transfemoral balloon wedge end hole catheter may be advanced across the major tricuspid orifice into the pulmonary artery and then used to exchange for the Wallstent/snare combination in the right ventricular outflow tract (RVOT) to serve as a target and capture system. Once inside the RVOT, the traversing guidewire may be snared and externalized through the femoral vein. As shown in FIG. 10B a target-capture device may be positioned into the RVOT to ensnare the guidewire that crossed from the coronary vein.

The cerclage rope 50 may be connected using heat-shrink tubing (polyether block amide, Cobalt Polymers, Cloverdale, California) to the guidewire and then pulled through the coronary sinus and interventricular septum into position. The distal tip of this guidewire may be transferred from the femoral to the subclavian sheath using a loop snare. As shown in FIG. 10C, the guidewire may be exchanged for the cerclage rope 50 containing an integrated arch portion 51 (arrowhead).

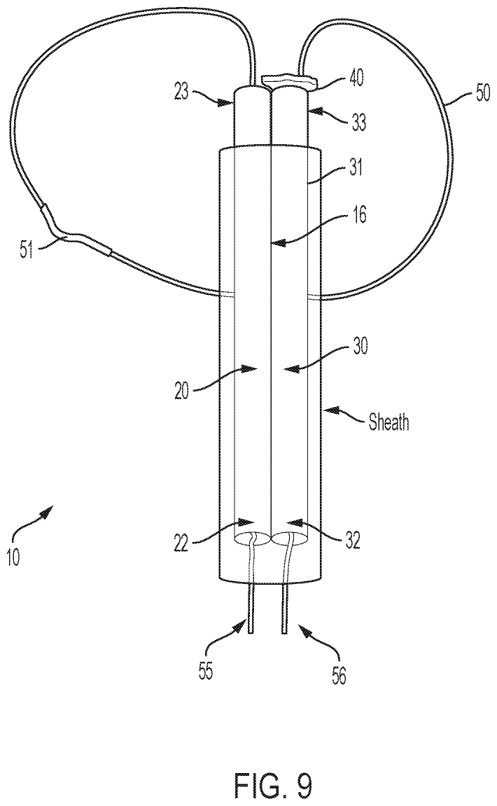

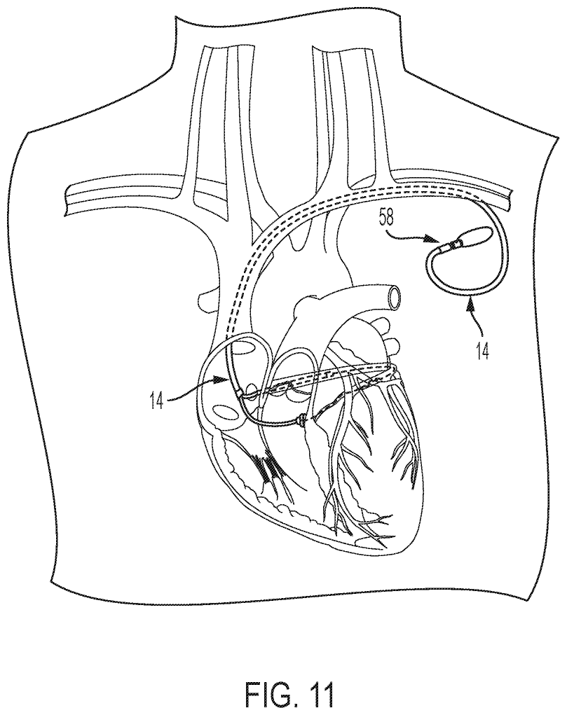

Next, the MLC device 10 may be advanced over the 2 free ends 55, 56 of the cerclage rope 50. FIG. 9 shows the straightened first and second arm portions 21, 31 of the first and second tubes 20, 30 contained within the sheath. Once the cerclage rope 50 is in place, the straightened first and second arm portions 21, 31 of the MLC device 10 is fed over the two ends 55, 56 of the cerclage rope 50 starting with its distal ends 23, 33 of the first and second tubes 20, 30. Once the MLC device 10 is in position within the heart, the first and second arm portions 21, 31 are restored into its preformed shapes as shown in FIG. 11.

Diagnostic coronary arteriography may allow fine positioning of the incorporated arched portion 51 of the cerclage rope 50. As shown in FIG. 10D during application of tension, the arch portion 51 may prevent compression of entrapped circumflex coronary artery branches.

Tension may be applied interactively during transthoracic echocardiography or TEE to achieve the intended reduction in mitral annular dimension and mitral valve regurgitation. As shown in FIG. 10E, in this caudal left anterior oblique projection, the MLC device 10 is shown to surround the mitral annular plane. Finally, the tension may be locked in the left subclavicular pocket and the skin incision closed. As shown in FIG. 10F, the tension locking device 58 may be embedded in the left subclavicular pocket

### MLC Transfemoral Procedure

The transfemoral procedure of MLC may also be feasible in addition to the aforementioned procedure. The trans-femoral procedure may include the following steps: (1) inserting a sheath introducer into the femoral vein; (2) inserting a guiding catheter to engage in the coronary sinus through the femoral sheath; (3) traversing 0.014″ guidewire into the interventricular septum, and exiting into the right ventricular (RV) cavity; (4) placing the wire catcher in the RV cavity through the femoral sheath and grabbing the exiting guidewire; (5) pulling the guidewire out to the femoral sheath with the catcher so that the guidewire makes up the path of the femoral vein: inferior vena cava (I VC)-coronary sinus (CS)-RV cavity-right atrium (RA)-IVC-femoral vein; (6) connecting the proximal end of the guidewire to cerclage rope 50 with a tension locking system 58; (7) replacing the guidewire with the cerclage rope 50 in place by pulling the distal end of the guidewire; (8) advancing the MLC device through the femoral sheath over the cerclage rope 50 with a delivery system; (9) when the MLC 10 is on optimal tension, securing the anchor 70 to keep the MLC in place; (10) when the MLC device is in place, a suitable tension is delivered through the tension lock device 58 under the guidance of real time imaging such as echocardiogram; (11) cutting the cerclage rope 50 by a special cutting device.

Advantages and features of the present invention, and a method of achieving them will become apparent with reference to the embodiments described below in detail together with the accompanying drawings. However, the present invention is not limited to the embodiments disclosed below and may be implemented in various different forms. The embodiments are provided to complete the disclosure of the present invention, and to completely inform the scope of the invention to those of ordinary skill in the art to which the present invention belongs, and the invention is only defined by the scope of the claims.

1. A method of treating mitral valve regurgitation in a patient, comprising:
(a) inserting a sheath introducer in a femoral vein;
(b) inserting a guide catheter to engage in a coronary sinus through a femoral sheath;
(c) traversing a guidewire into an interventricular septum and exiting into the right ventricular (RV) cavity;
(d) placing a guidewire catcher in the right ventricular cavity through the femoral sheath and grabbing the exiting guidewire;
(e) pulling the guidewire out to the femoral sheath with the guidewire catcher so that the guidewire makes up a path of the femoral vein: inferior vena cava (IVC)-coronary sinus (CS)-right ventricular (RV) cavity-right atrium (RA)-inferior vena cava (IVC)-femoral vein;
(f) connecting a proximal end of the guidewire to a cerclage rope with a tension lock device;
(g) replacing the guidewire with the cerclage rope in place by pulling a distal end of the guidewire;
(h) advancing an MLC device through the femoral sheath over the cerclage rope with a delivery system;
(i) when the MLC device is on optimal tension, securing an anchor to keep the MLC device in place;
(i) when the MLC device is in place, delivering a suitable tension through the tension lock device under a guidance of real time imaging such as echocardiogram;
(k) cutting the cerclage rope by a cutting device.
